(19) United States
(12) Patent Application Publication
(10) Pub. No.: US 2001/0036429 A1
(43) Pub. Date: Nov. 1, 2001

(54) Sterilization container

(75) Inventor: Lorenz Gabele, Sauldorf (DE)

Correspondence Address: LAW OFFICE OF BARRY R LIPSITZ, 755 MAIN STREET, MONROE, CT, 06468, US

(73) Assignee: Aesculap AG & Co. KG, Tuttlingen (DE)

(21) Appl. No.: 09/865,965

(22) Filed: May 25, 2001

Related U.S. Application Data
(63) Continuation of application No. PCT/EP98/08331, filed on Dec. 18, 1998.

(51) Int. Cl. A61L 2/00

(52) U.S. Cl. 422/292

(57) ABSTRACT

The aim of the invention is to permanently protect the surfaces of a sterilization container with a trough-shaped bottom part and a lid, which can be sealingly put onto said bottom part. To this end, the bottom part and/or the lid is/are coated with polytetrafluoroethylene.

The invention relates to a sterilization container comprising a trough-shaped lower part and a cover adapted to be sealingly placed thereon.

This type of sterilization container is used to contain surgical instruments; the instruments are sterilized in the container and before and thereafter stored. Containers of this type are usually made of metal, for example aluminum. For reasons of surface protection, and identification possibilities, the surfaces of such containers are often provided with a coating; preferably an anodized surface which if necessary can be colorized.

However, surfaces of this sort result in sterilization problems. Due to the aggressive sterilizing conditions, these surfaces undergo changes in their structure and color. Problems also occur when cleaning the sterilization container as anodized surfaces may only be treated using particular cleaning agents that are fundamentally different to those used in cleaning surgical instruments.

The object of the invention is to provide a generic sterilization container, the surfaces of which are not subject to the problems previously described.

This object is accomplished in a sterilization container of the kind described at the outset, in accordance with the invention, in that the lower part and/or the cover are coated with polytetrafluoroethylene.

This type of coating has proved surprisingly resistant, so that sterilization containers coated in this manner can withstand a large number of sterilization cycles with practically no occurrence of damage.

The coating can be applied to all surfaces, but in conformance with preferred embodiment it is possible that the lower part and/or the cover be coated only on the outside surfaces with polytetrafluoroethylene.

Additionally, the container hinge fittings and/or label tags can also be coated with polytetrafluoroethylene.

It is especially advantageous when a coloring agent is added to the polytetrafluoroethylene coating material. In this way, it is possible to color the whole, or parts of the container. This pigmented coating can likewise withstand the sterilization process with no change whatsoever, and even after 200 sterilization cycles such a container appears as new.

Furthermore, the coating has the advantage of possessing non-stick properties, so that contaminants will not adhere to the container surfaces. This is of great importance, especially for sterilization containers.

The sterilization container and component parts can be comprised of a variety of metals; it is however, especially advantageous when the metal is aluminum. This results in especially favorable sterilization characteristics, in particular, the, for sterilizing, especially favorable thermal conductibility and thermal capacity value of aluminum. Although aluminum surfaces suffer corrosive attack during the sterilization process, the polytetrafluoroethylene coating reliably protects these surfaces from any damage of that nature.

The following description of preferred embodiments of the invention serves in conjunction with the appended drawings to explain the invention in greater detail. The drawing shows:

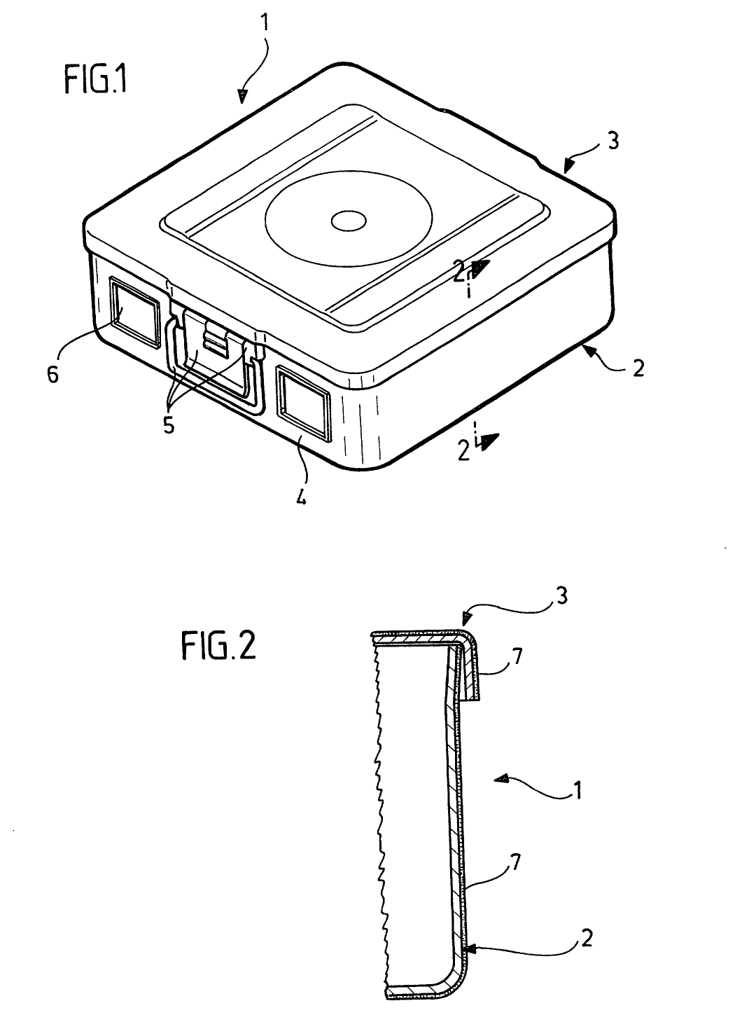

FIG. 1: a perspective view of the sterilization container and

FIG. 2: a sectional view along line 2-2 in FIG. 1

The sterilization container 1 depicted in the drawings comprises a trough-shaped bottom part 2 and a cover 3, adapted to be sealingly placed thereon, both should preferably be made of deep drawn aluminum.

Located on a sidewall (4) of the bottom part (2) are fittings (5), for example closing latch, lifting handle and their mountings, as well holding frames for label tags (6), on which information relating to the contents of the container etc. can be written.

The external surfaces of the cover (3) and the bottom part (2) are completely covered with a coating (7) of polytetrafluoroethylene, which may have a thickness of 3 μm to 50 μm but would preferably be between 15 μm to 20 μm thick. Pigments may be added to the polytetrafluoroethylene coating material thus enabling the cover and/or the bottom part to be colorized.

In the same way, individual or all of the fittings (5) and/or the label tags (6) can be polytetrafluoroethylene coated which if necessary may be pigmented.

The polytetrafluoroethylene coating possesses non-stick properties and thus repels contaminants, the coating makes the container more pleasing to handle, and surprisingly, such a coating has proved to be exceptionally robust, even when for example similarly coated containers are placed on top of each other. The resistance to abrasive wear and scratching is such that the container thereby suffers no damage. Finally, the coating reduces noise nuisance caused by containers striking together when stacking.

1. Sterilization container comprising a trough-shaped bottom part and a cover, adapted to be sealingly placed thereon, characterized in that the bottom part (2) and/or the cover (3) is coated with polytetrafluoroethylene.

2. Sterilization container as defined in claim 1, characterized in that the bottom part (2) and/or the cover (3) is coated on the external surfaces only with polytetrafluoroethylene.

3. Sterilization container as defined in claim 1, characterized in that the fittings (5) of the sterilization container (1) are coated with polytetrafluoroethylene.

4. Sterilization container as defined in claim 1, characterized in that the label tags (6) of the sterilization container (1) are coated with polytetrafluoroethylene.

5. Sterilization container as defined in claim 1, characterized in that pigments are added to the polytetrafluoroethylene coating material (7).

6. Sterilization container as defined in claim 2, characterized in that pigments are added to the polytetrafluoroethylene coating material (7).

7. Sterilization container as defined in claim 3, characterized in that pigments are added to the polytetrafluoroethylene coating material (7).

8. Sterilization container as defined in claim 4, characterized in that pigments are added to the polytetrafluoroethylene coating material (7).

9. Sterilization container as defined in claim 1, characterized in that the bottom part (2) and/or the cover (3) consists of aluminum.

10. Sterilization container as defined in claim 1, characterized in that the fittings (5) consist of aluminum.

11. Sterilization container as defined in claim 1, characterized in that the label tags (6) consist of aluminum.
